(19) United States
(12) Patent Application Publication
(10) Pub. No.: US 2025/0042832 A1
(43) Pub. Date: Feb. 6, 2025

(54) METHOD OF PREPARING GLUCARIC ACID FROM GLUCOSE USING NITROXIDE RADICAL-MEDIATED ORGANOCATALYST AND METHOD OF SEPARATING GLUCARIC ACID

(71) Applicant: SEOUL NATIONAL UNIVERSITY R&DB FOUNDATION, Seoul (KR)

(72) Inventors: In-Gyu CHOI, Seoul (KR); Jonghwa KIM, Seoul (KR); Jong-Chan KIM, Seoul (KR); Young-Min CHO, Seoul (KR); Chae-Hwi YOON, Seoul (KR); Daye KIM, Seoul (KR); Jun Ho SHIN, Seoul (KR); Hwanmyeong YEO, Seoul (KR); Hyo Won KWAK, Seoul (KR)

(21) Appl. No.: 18/442,238

(22) Filed: Feb. 15, 2024

(30) Foreign Application Priority Data
Jul. 31, 2023 (KR) 10-2023-0099909

(51) Int. Cl. C07C 51/31; B01J 27/08; B01J 31/02; C07C 51/487

(52) U.S. Cl. CPC C07C 51/316; B01J 27/08; B01J 31/0247; C07C 51/487

(57) ABSTRACT

Proposed is a method of preparing glucaric acid from glucose through TEMPO oxidation. The method of preparing glucaric acid includes (a) reacting glucose in a solvent through an oxidation reaction using 4-acetamido-TEMPO serving as a nitroxide radical-mediated organocatalyst, potassium bromide serving as a co-catalyst, and potassium hypochlorite serving as an oxidizing agent to prepare glucaric acid. The method can improve the conversion efficiency of glucose to glucaric acid through optimal conditions of the temperature, pH, and input of the oxidizing agent in an oxidation reaction using a TEMPO acid catalyst.

### CROSS REFERENCE TO RELATED APPLICATION

The present application claims priority to Korean Patent Application No. 10-2023-0099909, filed Jul. 31, 2023, the entire contents of which is incorporated herein for all purposes by this reference.

### BACKGROUND OF THE DISCLOSURE

### 1. Field of the Disclosure

The present disclosure relates to a method of preparing glucaric acid from glucose using a nitroxide radical-mediated organocatalyst and to a method of separating glucaric acid.

### 2. Description of the Related Art

Annual greenhouse gas emissions in Korea are rapidly increasing and are at a severe level, ranking 5th among OECD member countries (in 2017). In particular, according to carbon emissions in Korea by industry and sector, the petrochemical sector ranks second. Thus, the situation is that there is an urgent need to develop greenhouse gas reduction technology in the petrochemical industry. To cope with this situation, the government has prepared the “2050 Carbon Neutral Strategy” that simultaneously achieves net zero, economic growth, and improved quality of life for the people, and detailed tasks thereof are being derived and implemented.

Bioplastic, one of the countermeasures to reduce greenhouse gas emissions from the petrochemical industry, is defined as plastic produced from biomass, a carbon-neutral source. The domestic market of bioplastics has shown a trend in that disposable plastic products, including food packaging and industrial packaging, are being replaced with biomass-derived materials. Thus, the domestic market size of bioplastics is expected to grow faster in the future due to environmental regulations that are expected to be gradually strengthened in accordance with government policy in the future, increased public awareness, and the like.

Aldaric acid, one type of sugar acid, is an organic acid having two carboxyl groups through aldose oxidation and is a high value-added material applicable to various fields, such as detergents, metal complexing agents, and polymer monomers. Among these, glucaric acid derived from glucose is usable as a precursor of 2,5-furandicarboxylic acid (FDCA), the main monomer of polyethylene furanoate (PEF), a bioplastic to replace polyethylene terephthalate (PET). Additionally, glucaric acid is obtainable from glucose, the most abundant hexose in biomass, and is thus usable in food companies that can produce biomass-derived sugars. Additionally, glucaric acid is usable as a monomer of various polymers, such as polyester and polyamide, so it is expected that there will be demand for technology in the bioplastics industry capable of reducing carbon emissions.

Glucaric acid is more stable to heat and acid than 5-hydroxymethylfurfural(5-HMF), another precursor of FDCA, and thus is attracting attention as a precursor of FDCA that will overcome the instability of 5-HMF. Existing glucaric acid has been produced through the oxidation reaction of glucose using concentrated nitric acid or through a method of oxidizing glucose by introducing high-pressure oxygen in the presence of a precious metal catalyst. However, there are limitations in that concentrated nitric acid is harmful to the environment and can cause corrosion of equipment, and precious metal catalysts are based on expensive precious metals, which is problematic.

### SUMMARY OF THE INVENTION

The present disclosure, which has been made to solve the problems described above, aims to provide a method of preparing glucaric acid, that is, a highly stable raw material capable of overcoming the instability of 5-hydroxymethylfurfural (5-HMF), a raw material problematic in existing 2,5-furandicarboxylic acid (FDCA), and being produced from biomass-derived sugars.

Additionally, the present disclosure aims to provide a method of preparing glucaric acid, the method capable of improving the conversion efficiency of glucose to glucaric acid by using optimal combinations of the temperature, pH, and input of an oxidizing agent in an oxidation reaction using a nitroxide-based organocatalyst, for example, a TEMPO acid catalyst.

One aspect of the present disclosure provides a method of preparing glucaric acid, the method including reacting glucose in a solvent through an oxidation reaction using a nitroxide radical-mediated organocatalyst, a co-catalyst, and an oxidizing agent to prepare the glucaric acid or a salt thereof.

Additionally, the nitroxide radical-mediated organocatalyst may include one or more selected from the group consisting of 2,2,6,6-tetramethylpiperidine-1-oxyl (TEMPO), 4-acetamido-2,2,6,6-tetramethylpiperidine-1-oxyl (4-acetamido-TEMPO), 4-amino-2,2,6,6-tetramethylpiperidine-1-oxyl (4-amino-TEMPO), 4-carboxy-2,2,6,6-tetramethylpiperidine-1-oxyl (4-carboxy-TEMPO), 4-hydroxy-2,2,6,6-tetramethylpiperidine-1-oxyl benzoate (4-hydroxy-TEMPO benzoate), 4-hydroxy-2,2,6,6-tetramethylpiperidine-1-oxyl (4-hydroxy-TEMPO), 4-oxo-2,2,6,6-tetramethyl-1-piperidinyloxy (4-oxo-TEMPO), and di-tert-butyl nitroxide (DTBN).

Additionally, the nitroxide radical-mediated organocatalyst may include 4-acetamido-2,2,6,6-tetramethylpiperidine-1-oxyl (4-acetamido-TEMPO).

Additionally, the co-catalyst may include one or more selected from the group consisting of potassium bromide (KBr), sodium bromide (NaBr), sodium chloride (NaCl), and potassium chloride (KCl).

Additionally, the co-catalyst may include potassium bromide (KBr).

Additionally, the oxidizing agent may include one or more selected from the group consisting of potassium hypochlorite (KClO), sodium hypochlorite (NaClO), sodium chlorite (NaClO₂), potassium permanganate (KMnO₄), and potassium nitrate (KNO₃).

Additionally, the oxidizing agent may include potassium hypochlorite (KClO).

Additionally, the solvent may include one or more selected from the group consisting of water, acetonitrile, and dimethylformamide.

Additionally, the oxidation reaction may be performed at a temperature in a range of 0° C. to 20° C., which is preferably in the range of 2° C. to 15° C. and more preferably in the range of 3° C. to 10° C.

Additionally, the oxidation reaction may be performed at a pH in a range of 9 to 14, which is preferably in the range of 10 to 13.

Additionally, a ratio (m2/m1) of the number of moles of the oxidizing agent (m2) to the number of moles of glucose (m1) may be in a range of 1.5 to 6.0 (mol/mol) and is preferably in the range of 3.0 to 5.0 (mol/mol).

Another aspect of the present disclosure provides a method of separating glucaric acid, the method including: (a) reacting glucose in water serving as a solvent through an oxidation reaction using a nitroxide radical-mediated organocatalyst, potassium bromide, and potassium hypochlorite to prepare an aqueous solution containing glucaric acid or a potassium salt thereof; and (b) adding an acid to change the aqueous solution to an acidic aqueous solution, whereby glucaric acid or the potassium salt thereof is converted to a glucaric acid monopotassium salt and precipitated so that a mixture containing a precipitate of the glucaric acid monopotassium salt is obtained.

Additionally, the oxidation reaction may be performed at a pH in a range of 9 to 14, which is preferably in the range of 10 to 13.

Additionally, the acidic aqueous solution may have a pH in a range of 3 to 4.5, which is preferably in the range of 3.5 to 4.0.

Additionally, the acid may include one or more selected from the group consisting of hydrochloric acid, sulfuric acid, nitric acid, and bromic acid.

Additionally, the method of separating glucaric acid may further include, after (b): (c) reducing a temperature of the mixture to further precipitate the glucaric acid monopotassium salt; and (d) separating the glucaric acid monopotassium salt from the mixture.

Additionally, in (c), the temperature may be in a range of 1° C. to 10° C. and is preferably in the range of 2° C. to 6° C.

In the present disclosure, the instability of existing raw materials can be overcome because glucaric acid prepared from glucose can be produced from biomass-derived sugars and thus is highly stable.

Additionally, the conversion efficiency of glucose to glucaric acid can be improved through optimal conditions of temperature, pH, and input of an oxidizing agent in an oxidation reaction using a TEMPO acid catalyst.

### BRIEF DESCRIPTION OF THE DRAWINGS

These drawings are for the purpose of describing exemplary embodiments of the present disclosure, and therefore the technical idea of the present disclosure should not be construed as being limited to the accompanying drawings:

FIG. 1 is a schematic diagram illustrating a method of preparing glucaric acid from glucose according to Example 1 of the present disclosure;

FIG. 2 is a schematic diagram illustrating a conversion mechanism of glucose to glucaric acid through oxidation according to Example 1 of the present disclosure;

FIG. 3 is a schematic diagram illustrating a method of preparing glucaric acid from glucose according to Example 1 of the present disclosure;

FIG. 4 is a graph showing chromatography analysis results for analyzing the purity of a glucaric acid precipitate according to Example 1-1 of the present disclosure;

FIG. 5 is a graph showing nuclear magnetic resonance (NMR) spectroscopy analysis results for analyzing the structure of a glucaric acid precipitate according to Example 1-1 of the present disclosure;

FIG. 6 is a graph showing results of oxidizing glucose to glucaric acid through NaClO₂—NaClO-TEMPO oxidation with varying reaction temperatures according to Comparative Examples 1-1 and 1-2 of the present disclosure; and

FIG. 7 is a graph showing results of glucaric acid, gluconic acid, and oxalic acid yields through KClO-KBr-TEMPO oxidation with varying pH concentrations according to Examples 2-1 and 2-2 of the present disclosure.

### DESCRIPTION OF THE PREFERRED EMBODIMENTS

Hereinafter, embodiments of the present disclosure will be described in detail with reference to the accompanying drawings so that those skilled in the art can easily carry out the present disclosure.

However, the following description does not limit the present disclosure to specific embodiments. In the following description of the present disclosure, the detailed description of related arts will be omitted if it is determined that the gist of the present disclosure may be blurred.

Terms used herein are used only to describe specific embodiments and are not intended to limit the present disclosure. The singular expression includes the plural expression unless the context clearly indicates otherwise. It will be further understood that the terms “comprises”, “includes”, or “has” when used herein specify the presence of stated features, regions, integers, steps, operations, elements, and/or components, but do not preclude the presence or addition of one or more other features, regions, integers, steps, operations, elements, components, and/or combinations thereof.

Additionally, terms, such as “first”, “second”, etc. used herein, may be used to describe various components, but the components are not to be construed as being limited to the terms. These terms are used only for the purpose of distinguishing a component from another component. For example, without departing from the scope of the present disclosure, a first component may be referred to as a second component, and a second component may be also referred to as a first component.

Additionally, when a component is referred to as being “formed” or “laminated” on another component, it may be formed directly or attached to the front or one surface on the surface of the other component, but it will be understood that intervening elements may be present therebetween.

Hereinafter, a method of preparing glucaric acid from glucose using a nitroxide radical-mediated organocatalyst and a method of separating glucaric acid will be described in detail. However, these are disclosed only for illustrative purposes and not meant to limit the present disclosure, and the scope of the present disclosure is only defined by the appended claims.

One aspect of the present disclosure provides a method of preparing glucaric acid, the method including reacting glucose in a solvent through an oxidation reaction using a nitroxide radical-mediated organocatalyst, a co-catalyst, and an oxidizing agent to prepare the glucaric acid or a salt thereof.

Additionally, the nitroxide radical-mediated organocatalyst may include one or more selected from the group consisting of 2,2,6,6-tetramethylpiperidine-1-oxyl (TEMPO), 4-acetamido-2,2,6,6-tetramethylpiperidine-1-oxyl (4-acetamido-TEMPO), 4-amino-2,2,6,6-tetramethylpiperidine-1-oxyl (4-amino-TEMPO), 4-carboxy-2,2,6,6-tetramethylpiperidine-1-oxyl (4-carboxy-TEMPO), 4-hydroxy-2,2,6,6-tetramethylpiperidine-1-oxyl benzoate (4-hydroxy-TEMPO benzoate), 4-hydroxy-2,2,6,6-tetramethylpiperidine-1-oxyl (4-hydroxy-TEMPO), 4-oxo-2,2,6,6-tetramethyl-1-piperidinyloxy (4-oxo-TEMPO), and di-tert-butyl nitroxide (DTBN).

Additionally, the nitroxide radical-mediated organocatalyst may include 4-acetamido-2,2,6,6-tetramethylpiperidine-1-oxyl (4-acetamido-TEMPO).

Additionally, the co-catalyst may include one or more selected from the group consisting of potassium bromide (KBr), sodium bromide (NaBr), sodium chloride (NaCl), and potassium chloride (KCl).

Additionally, the co-catalyst may include potassium bromide (KBr).

Additionally, the oxidizing agent may include one or more selected from the group consisting of potassium hypochlorite (KClO), sodium hypochlorite (NaClO), sodium chlorite (NaClO₂), potassium permanganate (KMnO₄), and potassium nitrate (KNO₃).

Additionally, the oxidizing agent may include potassium hypochlorite (KClO).

Additionally, the solvent may include one or more selected from the group consisting of water, acetonitrile, and dimethylformamide.

Additionally, the oxidation reaction may be performed at a temperature in a range of 0° C. to 20° C., which is preferably in the range of 2° C. to 15° C. and more preferably in the range of 3° C. to 10° C. In this case, when performing the oxidation reaction at a temperature of lower than 0° C., the oxidation reaction fails to occur, which is undesirable. On the contrary, when performing the oxidation reaction at a temperature exceeding 20° C., the rapid reaction leads to the dissociation of glucaric acid, which is undesirable.

Additionally, the oxidation reaction may be performed at a pH in a range of 9 to 14, which is preferably in the range of 10 to 13. In this case, when performing the oxidation reaction at a pH of lower than 9, the oxidation reaction fails to occur easily, or side reactions lead to the generation of organic acids, which is undesirable. On the contrary, when performing the oxidation reaction at a pH exceeding 14, side reactions facilitate the generation of organic acids, which is undesirable.

Additionally, a ratio (m2/m1) of the number of moles of the oxidizing agent (m2) to the number of moles of glucose (m1) may be in a range of 1.5 to 6.0 (mol/mol) and is preferably in the range of 3.0 to 5.0 (mol/mol). In this case, when the ratio (m2/m1) of the number of moles of the oxidizing agent (m2) to the number of moles of glucose (m1) is lower than 1.5 (mol/mol), gluconic acid, an intermediate product of glucaric acid, is mainly produced, which is undesirable. On the contrary, when the ratio (m2/m1) of the number of moles of the oxidizing agent (m2) to the number of moles of glucose (m1) exceeds 6.0 (mol/mol), glucaric acid is dissociated and converted to organic acids, which is undesirable.

Another aspect of the present disclosure provides a method of separating glucaric acid, the method including: (a) reacting glucose in water serving as a solvent through an oxidation reaction using a nitroxide radical-mediated organocatalyst, potassium bromide, and potassium hypochlorite to prepare an aqueous solution containing glucaric acid or a potassium salt thereof; and (b) adding an acid to change the aqueous solution to an acidic aqueous solution, whereby glucaric acid or the potassium salt thereof is converted to a glucaric acid monopotassium salt and precipitated so that a mixture containing a precipitate of the glucaric acid monopotassium salt is obtained.

Additionally, the oxidation reaction may be performed at a pH in a range of 9 to 14, which is preferably in the range of 10 to 13. In this case, when performing the oxidation reaction at a pH of lower than 9, the oxidation reaction fails to occur easily, or side reactions lead to the generation of organic acids, which is undesirable. On the contrary, when performing the oxidation reaction at a pH exceeding 14, side reactions facilitate the generation of organic acids, which is undesirable.

Additionally, the acidic aqueous solution may have a pH in a range of 3 to 4.5, which is preferably in the range of 3.5 to 4.0. In this case, when the acidic aqueous solution has a pH of lower than 3, the potassium salt of the glucaric acid monopotassium salt is converted to glucaric acid (K salt converted to H), and thus the solubility in the aqueous solution increases, which is undesirable. On the contrary, when the acidic aqueous solution has a pH exceeding 4.5, the conversion to the glucaric acid monopotassium salt fails to be facilitated, and thus the precipitation yield decreases, which is undesirable.

Additionally, the acid may include one or more selected from the group consisting of hydrochloric acid, sulfuric acid, nitric acid, and bromic acid.

Additionally, the method of separating glucaric acid may further include, after (b): (c) reducing a temperature of the mixture to further precipitate the glucaric acid monopotassium salt; and (d) separating the glucaric acid monopotassium salt from the mixture.

Additionally, in (c), the temperature may be in a range of 1° C. to 10° C. and is preferably in the range of 2° C. to 6° C. In this case, when the temperature is lower than 1° C., organic acids may be precipitated in addition to the glucaric acid monopotassium salt, which is undesirable. On the contrary, when the temperature exceeds 10° C., the solubility of the glucaric acid monopotassium salt increases, and thus the precipitation yield decreases, which is undesirable.

### EXAMPLE

Hereinafter, the present disclosure will be described in more detail with examples. However, these examples are disclosed for illustrative purposes and the scope of the present disclosure is not limited thereby.

### Preparation Example 1: Aqueous Solution of Potassium Hypochlorite

A colloidal solution of calcium hypochlorite was prepared by adding 285.71 g of calcium hypochlorite to 600 ml of distilled water. An aqueous solution of potassium hydroxide and potassium carbonate was prepared by adding 40 g of potassium hydroxide and 140 g of potassium carbonate to 250 ml of distilled water. Then, the aqueous solution of potassium hydroxide and potassium carbonate was slowly added to the solution of calcium hypochlorite to vigorously stir the resulting mixture of calcium hypochlorite, potassium hydroxide, and potassium carbonate for about 30 minutes using a high-viscosity mechanical stirrer. After being stirred, a non-volatile phase, a gel-form impurity, was filtered out using filter paper, and the remaining liquid aqueous solution of potassium hypochlorite was extracted to prepare potassium hypochlorite, an oxidizing agent.

### Example 1: Preparation of Glucaric Acid Through TEMPO Oxidation

### Example 1-1: 5° C., 4.23 Times Input of Oxidizing Agent (Relative to Number of Moles of Glucose), pH 12

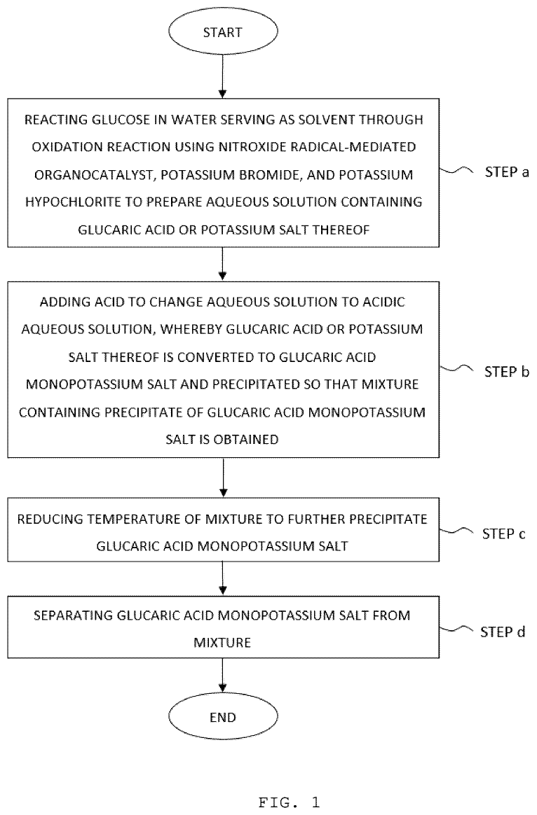

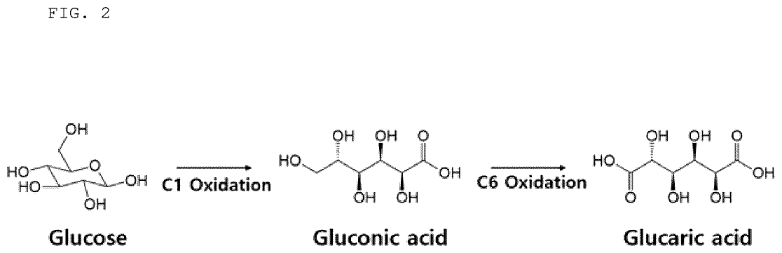

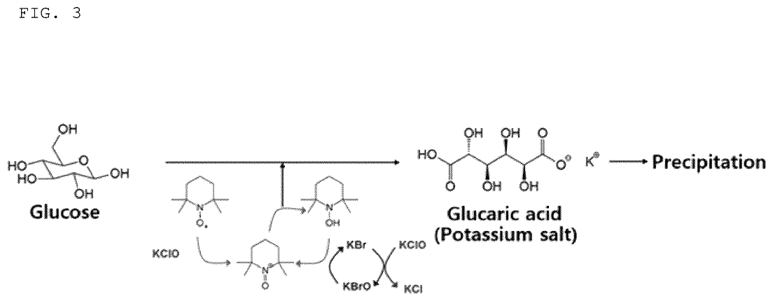

FIG. 1 is a schematic diagram illustrating a method of preparing glucaric acid from glucose according to Example 1 of the present disclosure, FIG. 2 is a schematic diagram illustrating a conversion mechanism of glucose to glucaric acid through oxidation according to Example 1 of the present disclosure, and FIG. 3 is a schematic diagram illustrating a method of preparing glucaric acid from glucose according to Example 1 of the present disclosure. Referring to FIGS. 1 to 3, a mixture was prepared by mixing a glucose standard material, 4-acetamido-TEMPO serving as a nitroxide-based organocatalyst, and potassium bromide serving as a co-catalyst. Then, potassium hypochlorite, serving as the oxidizing agent according to Preparation Example 1, was added to the prepared mixture at a rate of 0.2 ml/min. In this case, the oxidizing agent was added in an amount corresponding to 4.23 times relative to the number of moles of glucose to perform an oxidation reaction. The oxidation reaction was performed while maintaining the temperature at 5° C. and the pH at 12 using a 45% aqueous solution of potassium hydroxide. After completion of the oxidation reaction, concentrated hydrochloric acid was added to the resulting product containing glucaric acid to reduce the solubility of glucaric acid in water, thereby reducing the pH to 3.8 and converting glucaric acid to a glucaric acid monopotassium salt. The resulting product containing the glucaric acid monopotassium salt was refrigerated at a temperature of 4° C. for 24 hours to further reduce the solubility of glucaric acid monopotassium salt in water, thereby precipitating the glucaric acid monopotassium salt. Then, glucaric acid was prepared by separating the precipitated glucaric acid using filter paper.

### Examples 1-2 to 1-16

In Examples 1-2 to 1-16, glucaric acid was prepared in the same manner as in Example 1-1, except for applying oxidation temperatures, inputs of the oxidizing agent, and pH concentrations according to Table 1 below instead of the oxidation temperature of 5° C., the input of the oxidizing agent corresponding to 4.23 times (relative to the number of moles of glucose), and the pH of 12.

TABLE 1

| | | Input of oxidizing | |
|---|---|---|---|
| | | agent number of | |
| | Oxidation | moles of oxidizing | |
| | temperature | agent/number of | |
| Classification | (° C.) | moles of glucose) | pH |
| Example 1-1 | 5 | 4.23 | 12 |
| Example 1-2 | 0 | 1.65 | 10 |
| Example 1-3 | 10 | 1.65 | 10 |
| Example 1-4 | 0 | 4.95 | 10 |
| Example 1-5 | 10 | 4.95 | 10 |
| Example 1-6 | 0 | 1.65 | 13 |
| Example 1-7 | 10 | 1.65 | 13 |
| Example 1-8 | 0 | 4.95 | 13 |
| Example 1-9 | 10 | 4.95 | 13 |
| Example 1-10 | −3.41 | 3.3 | 11.5 |
| Example 1-11 | 13.41 | 3.3 | 11.5 |
| Example 1-12 | 5 | 0.53 | 11.5 |
| Example 1-13 | 5 | 6.08 | 11.5 |
| Example 1-14 | 5 | 3.3 | 9 |
| Example 1-15 | 5 | 3.3 | 14 |
| Example 1-16 | 5 | 3.3 | 11.5 |

### Example 2: Preparation of Glucaric Acid with Varying pH Concentrations

### Example 2-1: pH 2

A mixture was prepared by mixing a 10% (w/v) glucose standard material, 4-acetamido-TEMPO serving as a nitroxide-based organocatalyst, and potassium bromide (KBr) serving as a co-catalyst. Then, potassium hypochlorite (KClO), serving as the oxidizing agent according to Preparation Example 1, was added to the prepared mixture at a rate of 0.2 ml/min. The oxidizing agent was added in an amount corresponding to 4.23 times relative to the number of moles of glucose to perform an oxidation reaction. The oxidation reaction was performed while maintaining the temperature at 5° C. and the pH at 2. After completion of the oxidation reaction, glucaric acid was prepared by separating the precipitated glucaric acid using filter paper.

### Example 2-2: pH 7

In Example 2-2, glucaric acid was prepared in the same manner as in Example 2-1, except for maintaining the pH at 7 instead of 2.

### Comparative Example 1: Sodium Chlorite (NaClO₂)-Sodium Hypochlorite (NaClO)-TEMPO Oxidation

### Comparative Example 1-1: 35° C.

A mixture was prepared by adding a 1% (w/v) glucose standard material, a TEMPO acid catalyst serving as a nitroxide-based organocatalyst, and sodium hypochlorite (NaClO) serving as a co-catalyst to a solvent including acetonitrile and a phosphate buffer having a pH of 6.7 in a 1:1 volume ratio. Then, sodium chlorite (NaClO₂), serving as an oxidizing agent, was added to the prepared mixture at a rate of 0.2 ml/min. The oxidizing agent was added in an amount corresponding to 4 times relative to the number of moles of glucose to perform an oxidation reaction. The oxidation reaction was performed while maintaining the temperature at 35° C. and the pH at 12 using a 45% aqueous solution of potassium hydroxide. After completion of the oxidation reaction, glucaric acid was prepared by separating the precipitated glucaric acid using filter paper.

### Comparative Example 1-2: 45° C.

In Example 1-2, glucaric acid was prepared in the same manner as in Comparative Example 1-1, except for maintaining the temperature at 45° C. instead of 35° C.

### Experimental Example

### Experimental Example 1: Conversion Rate (%) to Glucaric Acid with Varying Oxidation Conditions

Table 2 below shows the conversion rates to glucaric acid, gluconic acid, formic acid, and oxalic acid with varying oxidation conditions. From Table 2, it was confirmed that the optimal oxidation conditions to achieve a high conversion rate to glucaric acid were as follows: an oxidation temperature of 5° C., an input of the oxidizing agent in an amount corresponding to 4.23 times relative to the number of moles of glucose, and a pH of 12, where 69.22% of glucaric acid relative to the number of moles of glucose was produced. Additionally, it was seen that while gluconic acid was produced as the main product when the input of the oxidizing agent was insufficient, glucaric acid, formic acid, and oxalic acid were produced as the main products when the input of the oxidizing agent increased. Additionally, oxalic acid and formic acid are produced when the C—C bond of gluconic acid is broken, where up to 3 units of oxalic acid and up to 6 units of formic acid may be generated from one molecule of gluconic acid. Thus, it was seen that when calculated in terms of molar yield, relatively high yields were exhibited. Additionally, it was confirmed that formic acid exhibited high yields under a condition where pH was 10, oxalic acid exhibited high yields under a condition where pH was about 13, and glucaric acid exhibited high yields under a condition where pH ranged from 11 to 12.

TABLE 2

| | | Input of | | Yield of oxides | | | |
|---|---|---|---|---|---|---|---|
| | | oxidizing agent | | (%, number of moles of | | | |
| | | (number of moles | | product/number of moles of | | | |
| | Oxidation | of oxidizing | | glucose ×100) | | | |
| | temperature | agent/number of | | Glucaric | Gluconic | Formic | Oxalic |
| Classification | (° C.) | moles of glucose) | pH | acid | acid | acid | acid |
| Example 1-1 | 5 | 4.23 | 12 | 69.22 | 0 | 25.46 | 14.13 |
| Example 1-2 | 0 | 1.65 | 10 | 3.81 | 68.54 | 1.41 | 0 |
| Example 1-3 | 10 | 1.65 | 10 | 7.77 | 87.49 | 1.56 | 0 |
| Example 1-4 | 0 | 4.95 | 10 | 25.23 | 11.07 | 5.62 | 77.43 |
| Example 1-5 | 10 | 4.95 | 10 | 38.24 | 5.61 | 6.78 | 65.79 |
| Example 1-6 | 0 | 1.65 | 13 | 11.28 | 90.54 | 5.27 | 0 |
| Example 1-7 | 10 | 1.65 | 13 | 16.7 | 96.18 | 7.56 | 0 |
| Example 1-8 | 0 | 4.95 | 13 | 60.47 | 7.5 | 32.21 | 10.63 |
| Example 1-9 | 10 | 4.95 | 13 | 68.45 | 5.91 | 56.49 | 13.03 |
| Example 1-10 | −3.41 | 3.3 | 11.5 | 64.59 | 22.67 | 9.44 | 16.43 |
| Example 1-11 | 13.41 | 3.3 | 11.5 | 7.51 | 29.34 | 17.04 | 12.13 |
| Example 1-12 | 5 | 0.53 | 11.5 | 0 | 62.24 | 0.5 | 0 |
| Example 1-13 | 5 | 6.08 | 11.5 | 20.48 | 3.52 | 16.33 | 88.47 |
| Example 1-14 | 5 | 3.3 | 9 | 4.93 | 37.12 | 1.93 | 0 |
| Example 1-15 | 5 | 3.3 | 14 | 2.13 | 76.66 | 70.02 | 0 |
| Example 1-16 | 5 | 3.3 | 11.5 | 64.11 | 25.67 | 12.53 | 14.71 |

### Experimental Example 2: Chromatography Analysis

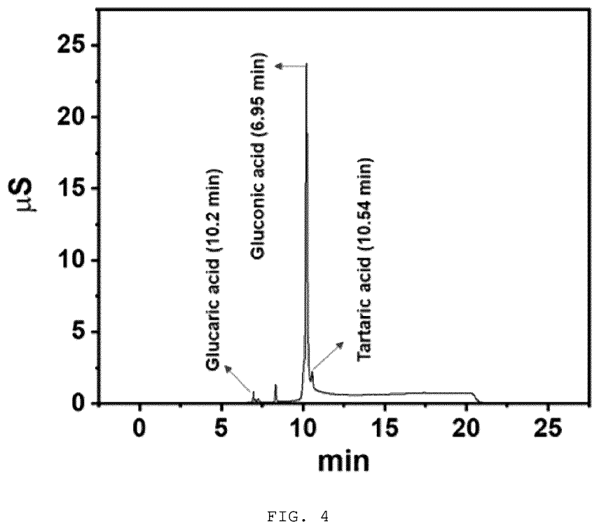

FIG. 4 is a graph showing the chromatography analysis results for analyzing the purity of a glucaric acid precipitate according to Example 1-1 of the present disclosure. From FIG. 4, showing the purity of the precipitate, it was seen that glucaric acid exhibited a purity of about 95.8%, and gluconic acid and tartaric acid were detected as the main by-products.

### Experimental Example 3: Nuclear Magnetic Resonance (NMR) Spectroscopy Analysis

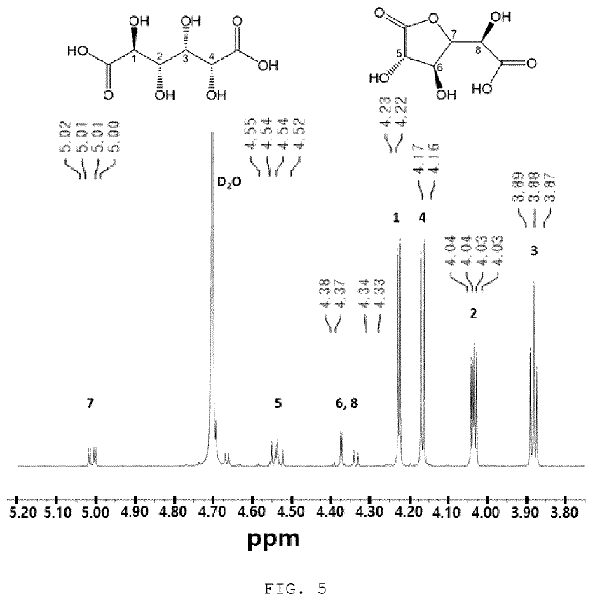

FIG. 5 is a graph showing nuclear magnetic resonance (NMR) spectroscopy analysis results for analyzing the structure of a glucaric acid precipitate according to Example 1-1 of the present disclosure. From FIG. 5, it was confirmed that glucaric acid and glucaric acid lactone having a lactone form in which hydroxyl and carboxyl groups in glucaric acid were esterified were detected. Such glucaric acid lactone is determined to be generated from lactonization caused by heat applied when dissolving glucaric acid in heavy water (D20) for NMR analysis of glucaric acid.

### Experimental Example 4: Chromatography Analysis

As a result of ion chromatography analysis for analyzing purity, it was confirmed that glucaric acid exhibited a purity of about 95.8%. In this case, gluconic acid and tartaric acid, an organic acid, were detected as the main by-products, and considering the purity of glucaric acid in the precipitate, about 67% of the produced glucaric acid was able to be recovered in a precipitate form.

### Experimental Example 5: Comparison of Glucaric Acid Yield Through NaClO₂—NaClO-TEMPO Oxidation with Varying Reaction Temperatures

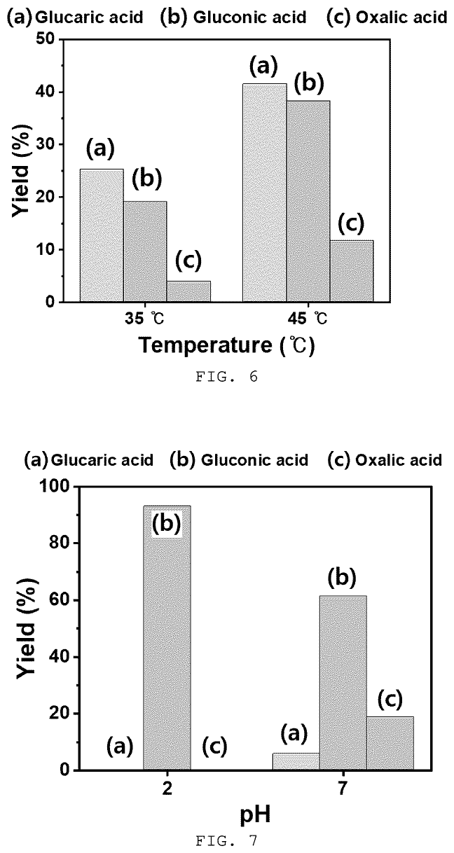

FIG. 6 is a graph showing the results of oxidizing glucose to glucaric acid through NaClO₂—NaClO-TEMPO oxidation with varying reaction temperatures according to Comparative Examples 1-1 and 1-2 of the present disclosure, and Table 3 below shows the glucaric acid, gluconic acid, and oxalic acid yields according to Comparative Examples 1-1 and 1-2. From FIG. 6 and Table 3, it was confirmed that glucaric acid sodium, whose solubility in water was high, was produced, making it challenging to separate and purify glucaric acid. Additionally, glucaric acid exhibited a yield of 25.32% at a temperature of 35° C. and a yield of 38.3% at a temperature of 45° C., relative to the number of moles of glucose, confirming that the oxidation reaction of glucose to glucaric acid through NaClO₂—NaClO-TEMPO exhibited low yields of glucaric acid.

TABLE 3

| | | Input of | | Yield of oxides | | |
|---|---|---|---|---|---|---|
| | | oxidizing agent | | (%, number of moles of | | |
| | | (number of moles | | product/number of moles of | | |
| | Oxidation | of oxidizing | | glucose ×100) | | |
| | temperature | agent/number of | | Glucaric | Gluconic | Oxalic |
| Classification | (° C.) | moles of glucose) | pH | acid | acid | acid |
| Comparative | 35 | 4 | 6.7 | 25.32 | 19.22 | 4.02 |
| Example 1-1 | | | | | | |
| Comparative | 45 | 4 | 6.7 | 38.3 | 41.58 | 11.77 |
| Example 1-2 | | | | | | |

### Experimental Example 6: Comparison of Glucaric Acid Yield Through KClO-KBr-TEMPO Oxidation with Varying pH Concentrations

FIG. 7 is a graph showing the results of glucaric acid, gluconic acid, and oxalic acid yields through KClO-KBr-TEMPO oxidation with varying pH concentrations according to Examples 2-1 and 2-2 of the present disclosure, and Table 4 below shows the glucaric acid, gluconic acid, and oxalic acid yields according to Examples 2-1 and 2-2. From FIG. 7 and Table 4, the yield of gluconic acid detected was 90% or more at a pH of 2. However, under the condition of pH 2, while glucose is converted to gluconic acid, gluconic acid is rarely converted to glucaric acid, confirming that glucaric acid is difficult to be prepared under the condition of pH 2.

Additionally, in the case of pH 7, the yield of glucaric acid is about 6%, showing a pattern that a portion of gluconic acid is converted to glucaric acid compared to the case of pH 2. However, the yield of gluconic acid is about 62%, confirming that the gluconic acid fails to be smoothly converted to glucaric acid and mostly remains as it is. Based on this confirmation, it is determined that while glucose is smoothly converted to gluconic acid at most pH, including acidic, neutral, and basic conditions, gluconic acid is smoothly converted to glucaric acid at alkaline conditions, especially at a pH concentration of about 12.

TABLE 4

| | | Input of | | Yield of oxides | | |
|---|---|---|---|---|---|---|
| | | oxidizing agent | | (%, number of moles of | | |
| | | (number of moles | | product/number of moles of | | |
| | Oxidation | of oxidizing | | glucose ×100) | | |
| | temperature | agent/number of | | Glucaric | Gluconic | Oxalic |
| Classification | (° C.) | moles of glucose) | pH | acid | acid | acid |
| Example 2-1 | 5 | 4.23 | 2 | 0 | 93.15 | 0.3 |
| Example 2-2 | 5 | 4.23 | 7 | 6.04 | 61.57 | 18.91 |

Although preferred embodiments of the present disclosure have been disclosed for illustrative purposes, those skilled in the art will appreciate that diverse variations and modifications are possible through addition, alteration, deletion, etc. of elements, without departing from the spirit and scope of the present disclosure. For example, each component described as a single type may be implemented to be distributed and similarly, components described to be distributed may also be implemented in an associated form. The scope of the present disclosure is defined by the appended claims rather than the detailed description presented above. All changes or modifications derived from the meaning and scope of the claims and the concept of equivalents should be construed to fall within the scope of the present disclosure.

What is claimed is:

1. A method of preparing glucaric acid, the method comprising reacting glucose in a solvent through an oxidation reaction using a nitroxide radical-mediated organocatalyst, a co-catalyst, and an oxidizing agent to prepare the glucaric acid or a salt thereof.

2. The method of claim 1, wherein the nitroxide radical-mediated organocatalyst comprises one or more selected from the group consisting of 2,2,6,6-tetramethylpiperidine-1-oxyl (TEMPO), 4-acetamido-2,2,6,6-tetramethylpiperidine-1-oxyl (4-acetamido-TEMPO), 4-amino-2,2,6,6-tetramethylpiperidine-1-oxyl (4-amino-TEMPO), 4-carboxy-2,2,6,6-tetramethylpiperidine-1-oxyl (4-carboxy-TEMPO), 4-hydroxy-2,2,6,6-tetramethylpiperidine-1-oxyl benzoate (4-hydroxy-TEMPO benzoate), 4-hydroxy-2,2,6,6-tetramethylpiperidine-1-oxyl (4-hydroxy-TEMPO), 4-oxo-2,2,6,6-tetramethyl-1-piperidinyloxy (4-oxo-TEMPO), and di-tert-butyl nitroxide (DTBN).

3. The method of claim 2, wherein the nitroxide radical-mediated organocatalyst comprises 4-acetamido-2,2,6,6-tetramethylpiperidine-1-oxyl (4-acetamido-TEMPO).

4. The method of claim 1, wherein the co-catalyst comprises one or more selected from the group consisting of potassium bromide (KBr), sodium bromide (NaBr), sodium chloride (NaCl), and potassium chloride (KCl).

5. The method of claim 4, wherein the co-catalyst comprises potassium bromide (KBr).

6. The method of claim 1, wherein the oxidizing agent comprises one or more selected from the group consisting of potassium hypochlorite (KClO), sodium hypochlorite (NaClO), sodium chlorite (NaClO₂), potassium permanganate (KMnO₄), and potassium nitrate (KNO₃).

7. The method of claim 6, wherein the oxidizing agent comprises potassium hypochlorite (KClO).

8. The method of claim 1, wherein the solvent comprises one or more selected from the group consisting of water, acetonitrile, and dimethylformamide.

9. The method of claim 1, wherein the oxidation reaction is performed at a temperature in a range of 0° C. to 20° C.

10. The method of claim 1, wherein the oxidation reaction is performed at a pH in a range of 9 to 14.

11. The method of claim 1, wherein a ratio (m2/m1) of the number of moles of the oxidizing agent (m2) to the number of moles of glucose (m1) is in a range of 1.5 to 6.0 (mol/mol).

12. A method of separating glucaric acid, the method comprising:
(a) reacting glucose in water serving as a solvent through an oxidation reaction using a nitroxide radical-mediated organocatalyst, potassium bromide, and potassium hypochlorite to prepare an aqueous solution containing glucaric acid or a potassium salt thereof; and
(b) adding an acid to change the aqueous solution to an acidic aqueous solution, whereby glucaric acid or the potassium salt thereof is converted to a glucaric acid monopotassium salt and precipitated so that a mixture comprising a precipitate of the glucaric acid monopotassium salt is obtained.

13. The method of claim 12, wherein the oxidation reaction is performed at a pH in a range of 9 to 14.

14. The method of claim 12, wherein the acidic aqueous solution has a pH in a range of 3 to 4.5.

15. The method of claim 12, wherein the acid comprises one or more selected from the group consisting of hydrochloric acid, sulfuric acid, nitric acid, and bromic acid.

16. The method of claim 12, further comprising, after the (b):
(c) reducing a temperature of the mixture to further precipitate the glucaric acid monopotassium salt; and
(d) separating the glucaric acid monopotassium salt from the mixture.

17. The method of claim 16, wherein in the (c), the temperature is in a range of 1° C. to 10° C.
